(19) United States
(12) Patent Application Publication
(10) Pub. No.: US 2025/0001052 A1
(43) Pub. Date: Jan. 2, 2025

(54) COATED EPTFE LINERS FOR CATHETERS

(71) Applicant: Zeus Company LLC, ORANGEBURG, SC (US)

(72) Inventors: Alexandra Nijela Younan, Columbia, SC (US); John Richard Campanelli, We, SC (US)

(21) Appl. No.: 18/754,694

(22) Filed: Jun. 26, 2024

Related U.S. Application Data
(60) Provisional application No. 63/523,427, filed on Jun. 27, 2023.
(60) Provisional application No. 63/638,058, filed on Apr. 24, 2024.

(51) Int. Cl. A61L 29/08; A61L 29/04; A61M 25/00

(52) U.S. Cl. CPC A61L 29/085; A61L 29/041; A61M 25/0045; A61M 25/005

(57) ABSTRACT

Expanded polytetrafluoroethylene (ePTFE) tubings are provided with a thermoplastic tie layer coating on the outer surface thereof to serve, e.g., as flexible liners for catheters. The thermoplastic tie layer coating can be anchored into the pores of the ePTFE but advantageously does not permeate through to the inner surface of the tubing. In this way, the outer surface of the tubing can adhere to an outer jacket material while the inner surface of the tubing remains lubricious.

### CROSS-REFERENCE TO RELATED APPLICATIONS

This application claims priority to U.S. Provisional Patent Application Ser. Nos. 63/523,427, filed Jun. 27, 2023 and 63/638,058, filed Apr. 24, 2024, each of which is incorporated herein by reference in its entirety.

### FIELD OF THE DISCLOSURE

The present disclosure relates to tubings comprising tie layer coatings thereon, which tubings can serve as components of catheter assemblies.

### BACKGROUND

Polytetrafluoroethylene (PTFE) has been an ideal material for inner liners of catheters due to the chemical resistance, biocompatibility, and low coefficient of friction (COF) of PTFE. PTFE exhibits unique characteristics in this field that other polymers do not exceed. With such a low COF, PTFE has been able to provide an inner diameter (ID) that easily allows various catheter technologies such as stents, balloons, atherectomy or thrombectomy devices to be pushed through a small diameter catheter lumen. The effect of high lubricity of the catheter ID is a reduced deployment force of catheter devices as the catheter devices are passed through the lumen of the catheter ID, increasing the likelihood of a successful procedure. In catheter constructions, the catheter liner is stretched over a mandrel, which usually comprises stainless steel or PTFE. A braided or coiled wire reinforcing layer may be constructed on top of the liner and can vary in picks, wire dimensions, and materials for different applications. A catheter jacket is then slid over the underlying layers, followed by a heat shrink tube over the catheter jacket. A thermoplastic tie layer made typically of the same material and durometer as the catheter jacket can be deposited on the surface of the catheter liner for enhanced adhesion. The finalized construction is then laminated together and removed from the mandrel, resulting in a fully built catheter.

As minimally invasive surgical procedures using catheters extend further into the vasculature, such as above the neck or below the knee applications, catheters must navigate smaller vessels and sharper turns. In neurovascular procedures to treat ischemic strokes, catheters are required to deliver therapies in a safe and effective manner through tortuous blood vessels. These catheter liners are thus required to be highly flexible in addition to being highly lubricious.

Thus, development of catheter liners capable of providing both surface lubricity and flexibility would be desirable in the art.

### SUMMARY

A catheter liner is provided based on an ePTFE tubing with a thermoplastic tie layer coated on at least a portion of an outer surface thereof. The liner is a thin-walled ePTFE tubing that advantageously has a relatively open node and fibril structure, and therefore a relatively large internodal distance (IND), to allow the tie layer material to better adhere to the outer surface of the ePTFE tubing. The thermoplastic tie layer coating can comprise or consist essentially of one or more of PEBA, nylon, and/or polyurethane (as well as derivatives and/or copolymers thereof).

In some embodiments, the IND of the ePTFE tube is between 5 μm and 100 μm. In some embodiments, the IND of the ePTFE tube is essentially constant throughout the wall thickness from ID to outer diameter (OD). In some embodiments, the IND of the ePTFE tube is greater on the OD and smaller on the ID.

The disclosure further provides methods of preparing coated ePTFE tubings, e.g., coated ePTFE liners. In one non-limiting embodiment, an ePTFE tubing is placed on a mandrel and dipped at an angle into a solution of the thermoplastic. The coated tube is then removed from the solution and removed from the mandrel and dried, forming the thermoplastic-coated surface of the tubing/catheter liner of the disclosure. The conditions of the dip coating process can, in some embodiments, be altered to create customized coating thickness and properties of the ePTFE-based catheter liner.

In some embodiments, the thermoplastic tie layer coating thickness is between 1 μm and 100 μm. In some embodiments the coated ePTFE tubing is prepared using a thermoplastic solution comprising 5% PEBA 55D in n-butanol. In some embodiments, the thermoplastic solution is 5% PEBA 25D in n-butanol. In some embodiments, the thermoplastic solution is 5% TECOFLEX® 93A polyurethane in n-butanol. In some embodiments, the thermoplastic solution is 3% TECOFLEX® 93A polyurethane in n-butanol. In some embodiments, the thermoplastic solution is 8% TECOTHANE® polyurethane in tetrahydrofuran. In some embodiments, the solution is at room temperature. In some embodiments, the solution is heated up to 10° C. below the solvent boiling point. In some embodiments, the durometer hardness of the thermoplastic resin used to prepare the disclosed coated ePTFE tubings as tested according to ASTM D2240 is between 93A and 55D. In some embodiments, the coated tube is dried in air at room temperature. In some embodiments, the coated tube is dried at elevated temperature, such as in a circulating air oven at temperatures e.g., as high as the boiling point of the solvent.

In some embodiments, the amount of time the ePTFE liner is submerged in the thermoplastic solution is between 10 and 60 seconds. In some embodiments, the ePTFE liner is dip coated and dried before performing an additional dip coating process. In some embodiments, the coated tube is dried in air at room temperature. In some embodiments, the tube is dried in a circulating air oven at temperatures as high as the boiling point of the solvent. In some embodiments, the durometer hardness of the thermoplastic resin (following application) as tested according to ASTM D2240 is between 93A and 55D.

The invention includes, without limitation, the following embodiments.
Embodiment 1: A coated tubing, comprising: an expanded polytetrafluoroethylene (ePTFE) tubing with an inner surface and an outer surface and a wall with a thickness; and a thermoplastic tie layer coating on at least a portion of the outer surface.
Embodiment 2: The coated tubing of Embodiment 1, wherein the thermoplastic tie layer coating is on substantially all of the outer surface.
Embodiment 3: The coated tubing of Embodiment 1 or 2, wherein the thermoplastic tie layer coating permeates into the wall from the outer surface through the wall of the ePTFE tubing to a depth of less than the wall thickness. Embodiment 4: The coated tubing of any of Embodiments 1-3, wherein the thermoplastic tie layer permeates into the wall from the outer surface through the wall of the ePTFE tubing to a depth of 80% or less of the wall thickness, 70% or less of the wall thickness, or 60% or less of the wall thickness.
Embodiment 5: The coated tubing of any of Embodiments 1-4, wherein the thermoplastic tie layer permeates into the wall from the outer surface through the wall of the ePTFE tubing to a depth of 50% or less of the wall thickness, 40% or less of the wall thickness, or 30% or less of the wall thickness.
Embodiment 6: The coated tubing of any of Embodiments 1-5, wherein the inner surface comprises substantially no thermoplastic tie layer coating or no thermoplastic tie layer coating.
Embodiment 7: The coated tubing of any of Embodiments 1-6, wherein the thermoplastic tie layer coating has an average coating thickness from the outer surface outwards of 1 μm or greater.
Embodiment 8: The coated tubing of any of Embodiments 1-7, wherein the thermoplastic tie layer coating has a durometer hardness between 93A and 55D.
Embodiment 9: The coated tubing of any of Embodiments 1-8, wherein the thermoplastic tie layer coating comprises or consists essentially of one or more of a polyether block amide, a polyurethane, a nylon, or a derivative or copolymer thereof.
Embodiment 10: The coated tubing of any of Embodiments 1-9, wherein the thermoplastic tie layer coating is non-uniformly distributed throughout the wall thickness.
Embodiment 11: The coated tubing of any of Embodiments 1-10, wherein the ePTFE tubing has an inner diameter of 0.6 mm or greater.
Embodiment 12: The coated tubing of any of Embodiments 1-11, wherein the ePTFE tubing has an average wall thickness of 0.03 mm or greater, 0.04 mm or greater, or 0.05 mm or greater, e.g., 0.03 to 0.1 mm.
Embodiment 13: The coated tubing of any of Embodiments 1-12, wherein the ePTFE tubing has an internodal distance (IND) of greater than 5 μm.
Embodiment 14: A catheter comprising the coated tubing of any of Embodiments 1-13, configured such that the catheter has a catheter inner surface corresponding to the inner surface of the ePTFE tubing.
Embodiment 15: The catheter of Embodiment 14, further comprising a reinforcing layer on an outer surface of the coated tubing (e.g., a braided or coiled wire).
Embodiment 16: The catheter of Embodiment 14 or 15, further comprising a catheter jacket.
Embodiment 17: A catheter assembly comprising: an expanded polytetrafluoroethylene (ePTFE) liner with an inner surface and an outer surface and a wall thickness; and a thermoplastic tie layer coating with an average coating thickness of 1 μm or greater and a durometer hardness between 93A and 55D disposed on the outer surface of the ePTFE liner to give a coated liner, wherein the ePTFE liner has a bonding affinity to the thermoplastic tie layer coating.
Embodiment 18: The catheter assembly of Embodiment 17, wherein the thermoplastic tie layer coating is on substantially all of the outer surface.
Embodiment 19: The catheter assembly of Embodiment 17 or 18, wherein the thermoplastic tie layer coating permeates into the wall from the outer surface through the wall of the ePTFE tubing to a depth of less than the wall thickness.
Embodiment 20: The catheter assembly of claim 19, wherein the thermoplastic tie layer coating permeates into the wall to a depth of 80% or less of the wall thickness, 60% or less of the wall thickness, or 50% or less of the wall thickness.
Embodiment 21: The catheter assembly of any of Embodiments 17-20, wherein the inner surface comprises no thermoplastic tie layer coating.
Embodiment 22: The catheter assembly of any of Embodiments 17-21, wherein the thermoplastic tie layer coating has an average coating thickness from the outer surface outwards of 1 μm or greater.
Embodiment 23: The catheter assembly of any of Embodiments 17-22, wherein the thermoplastic tie layer coating comprises or consists essentially of one or more of a polyether block amide, a polyurethane, a nylon, or a derivative or copolymer thereof.
Embodiment 24: The catheter assembly of any of Embodiments 17-23, wherein the thermoplastic tie layer coating is non-uniformly distributed throughout the wall thickness.
Embodiment 25: The catheter assembly of any of Embodiments 17-24, wherein the ePTFE liner has an inner diameter of 0.6 mm or greater.
Embodiment 26: The catheter assembly of any of Embodiments 17-25, wherein the ePTFE liner has an average wall thickness of 0.03 mm or greater, 0.04 mm or greater, or 0.05 mm or greater, e.g., 0.03 to 0.1 mm.
Embodiment 27: The catheter assembly of any of Embodiments 17-26, wherein the ePTFE liner has an internodal distance of greater than 5 μm.
Embodiment 28: The catheter assembly of any of Embodiments 17-27, further comprising a reinforcing layer on an outer surface of the thermoplastic tie layer coating (e.g., a braided or coiled wire).
Embodiment 29: The catheter assembly of any of Embodiments 17-28, further comprising a catheter jacket.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

Having thus described the disclosure in the foregoing general terms, reference will now be made to the accompanying drawings, which are not necessarily drawn to scale, and wherein:

FIG. 1 is a non-limiting general schematic of a tube, showing thickness of the wall, “T” inner diameter “ID” and outer diameter “OD”;

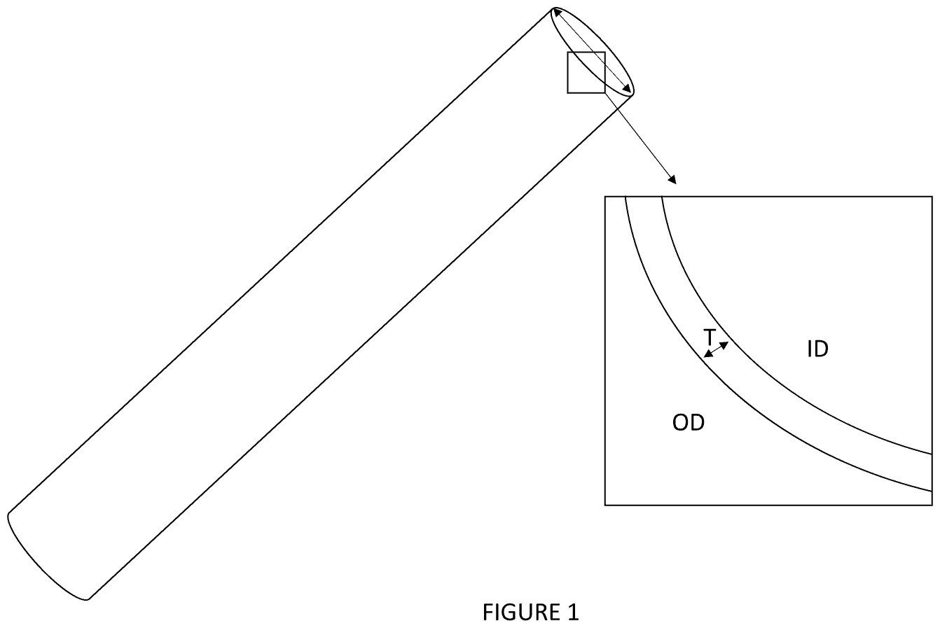

FIG. 2 is schematic illustrations of certain non-limiting coated tubes (a-d) with varying degrees of penetration of the coating into the tube walls, according to the disclosure, with varying levels of penetration of the coating layer into the tube wall;

FIG. 3 is a scanning electron microscopy (“SEM”) micrograph at 1000× magnification of the tube of Example 3;

FIG. 4 is SEM Micrograph at 1000× magnification of the tube of Example 4;

FIG. 5 is a SEM Micrograph at 1000× magnification of the tube of Comparative Example 1; and

FIG. 6 is an image of: a) a non-limiting ePTFE liner with a green-dyed PEBA 55D tie layer on glass-filled PTFE mandrel; and b) a non-limiting ePTFE liner with green PEBA 55D tie layer slit open on a glass-filled PTFE mandrel.

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise.

The present disclosure generally provides coated tubes, e.g., with use as catheter liners, which comprise expanded PTFE (“ePTFE”) tubings and one or more thermoplastic tie layer coatings on at least a portion of an outer surface thereof (e.g., including on substantially all or all of the outer surface thereof). “Substantially all” in this context can include, e.g., at least 90% of the surface area of the outer surface, at least 95% of the surface area of the outer surface, at least 98% of the surface area of the outer surface, or at least 99% of the surface area of the outer surface, up to e.g., 100% of the surface area of the outer surface.

The ePTFE tubing which is a component of the disclosed coated tubes according to the present disclosure is not particularly limited. In some embodiments, the ePTFE tubing is manufactured according to U.S. Pat. No. 3,953,566 to Gore, which is incorporated herein by reference in its entirety. Porous expanded PTFE tubing is generally extruded from a mixture of PTFE fine powder and lubricant and then stretched such that the microstructure of the tubing contains nodes that are connected by fibrils. This process provides tubes that are a softer, more flexible porous alternative to standard PTFE. As will be provided in further detail herein, in some embodiments, ePTFE tubings can be prepared and used as a flexible catheter liner, utilizing a thermoplastic tie layer coating to adhere to the catheter jacket and thus the rest of the catheter construction (as provided herein).

The dimensions of the ePTFE tubing can vary. Dimensions can be measured, e.g., by micrometer, diameter pins, digital microscope, or scanning electron microscope. In some embodiments, the ePTFE tubing is considered to be a “thin-walled ePTFE tubing.” In some embodiments, the ePTFE tubing has an average wall thickness of 0.03 mm or more, about 0.04 mm or more (e.g., 0.041 mm or more), or about 0.05 mm or more. In some embodiments, the ePTFE tubing has an average wall thickness of 0.1 mm or less, about 0.25 mm or less, about 0.2 mm or less, about 0.1 mm or less, or about 0.08 mm or less. In some embodiments, the ePTFE tubing has an inner diameter (“ID”) of about 0.6 mm or more, e.g., about 0.6 mm to about 5 mm or about 0.6 mm to about 3 mm in some embodiments.

In some embodiments, the internodal distance (IND) of the ePTFE has a value exceeding 5 μm, exceeding 6 μm, exceeding 7 μm, exceeding 8 μm, exceeding 9 μm, or exceeding 10 μm. For example, the IND can be about 5 μm or about 10 μm to about 20 μm, about 50 μm, about 70 μm, or about 100 μm. According to the present disclosure, tubings comprising ePTFE with a relatively high IND (e.g., within the above referenced ranges) exhibit a relatively open node and fibril structure, which allows the thermoplastic tie layer coating to better adhere to the outer surface of the ePTFE tubing. It is noted that, in some embodiments, the IND of the ePTFE tube is substantially constant throughout the wall thickness (from ID to OD). In some embodiments, the IND of the ePTFE tube is inconsistent throughout the wall thickness (e.g., having a greater IND on the OD and a smaller IND on the ID or vice versa). IND of an ePTFE tubing can be measured, for example, by slitting the tube open and laying it flat on the stage of a scanning electron microscope (SEM) with the outer surface (OD) of the tube facing upward. The IND can then be measured node-to-node parallel to the fibrils using the SEM. In some embodiments, a method of producing a coated ePTFE tubing is provided, which first comprises a step of characterizing the suitability of an ePTFE tubing for subsequent coating via the method above, e.g., to confirm that the IND meets or exceeds the referenced values to ensure for sufficient adherence of the thermoplastic tie layer material to the surface.

In some embodiments, the ePTFE tubing comprises an untreated (e.g., unetched) surface. While it is common to use adhesive material or physical/chemical pre-treatments (e.g., plasma treating or chemical etching) prior to applying coatings on PTFE-based materials to ensure sufficient bonding, in some embodiments, such pre-treatments are not employed in the context of the present disclosure. Although such processes are not excluded from the present disclosure, in some embodiments, it may be advantageous to avoid such processes (which may be destructive to the node and fibril structure of an ePTFE liner). As such, in some embodiments, the coated tubings, liners, and constructions provided herein comprise no adhesive material and/or do not comprise etched ePTFE surfaces.

The thermoplastic coating of the disclosed coated tubes can vary. In some embodiments, the thermoplastic coating comprises or consists essentially of one or more thermoplastic materials, including, but not limited to, thermoplastic elastomers. In some embodiments, the thermoplastic coating comprises or consists essentially of one or more of polyether block amide (PEBA), nylon, and/or polyurethane (PU). PEBA can be, for example, PEBAX® from Arkema or VESTAMID® E from Evonik Industries. Nylon can be, for example, any of the various types of nylons known, including, but not limited to, nylon 1, 6, nylon 4, 6, nylon 6, nylon 6,6, nylon 6/10, and nylon 6/12. Polyurethane can be, for example, TECOFLEX™ polyurethanes from Lubrizol.

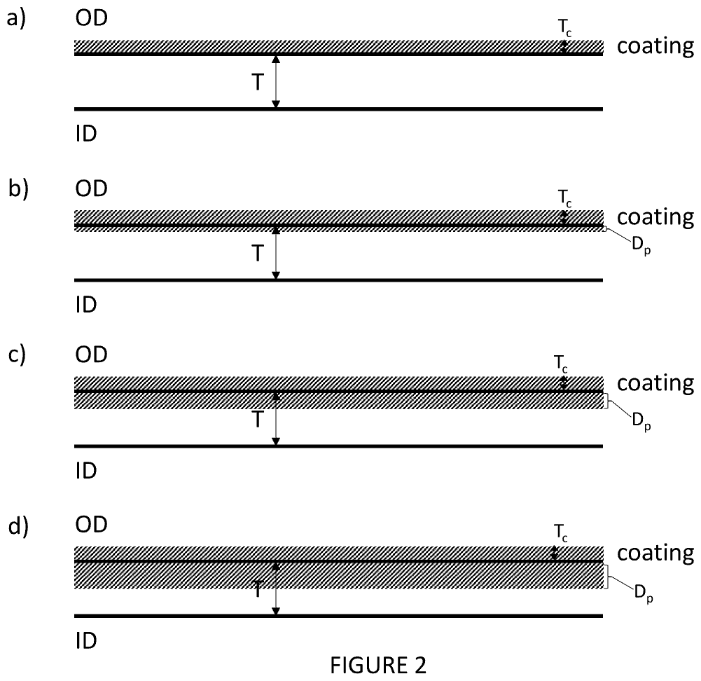

The thermoplastic coating can be of varying thicknesses (T_{c}) on the OD of the tubing, as shown, e.g., in FIG. 2 (from the OD of the tubing outwards away from the lumen of the tubing). The average thickness of the thermoplastic coating in some embodiments can be, e.g., about 1 μm or greater. In some embodiments, the thickness of the thermoplastic coating can be, e.g., about 1 μm to about 100 μm, including about 1 μm to about 75 μm, about 1 μm to about 50 μm, about 1 μm to about 25 μm, about 1 μm to about 10 μm, about 10 μm to about 100 μm, about 10 μm to about 75 μm, about 10 μm to about 50 μm, about 10 μm to about 25 μm, about 25 μm to about 100 μm, about 25 μm to about 75 μm, about 25 μm to about 50 μm, about 50 μm to about 100 μm, about 50 μm to about 75 μm, or about 75 μm to about 100 μm.

In some embodiments, the thermoplastic coating material can be described as being substantially (or only) present on the outer surface of the catheter liner. By “substantially” or “only” present on the outer surface of the catheter liner is meant that no significant amount of thermoplastic material is present on the inner surface (ID) of the tubing. However, as provided herein, thermoplastic coating material may be present, to some extent, within the walls of the ePTFE tubing. ePTFE tubings according to the present disclosure may, in some embodiments, be coated on the outer surface such that the thermoplastic material permeates/penetrates partially into the ePTFE wall without having the material permeate fully through the wall into the inner surface of the tubing. In this way, the inner surface of the tubing (ID) can advantageously be substantially or fully uncoated ePTFE (i.e., the inner surface of the coated tubing consists essentially of ePTFE). The outer surface of the tubing (OD) can, in some such embodiments, consist essentially of the thermoplastic coating material. In some embodiments, the thermoplastic coating material exhibits a physical surface bond between nodes and fibrils of the ePTFE, but does not significantly impact/destroy the microstructure of the ePTFE (e.g., with little to no thermoplastic coating material on the inner surface of the tubing).

In some embodiments, e.g., as shown in FIG. 2 a), the thermoplastic material coating does not penetrate to any significant extent into the tubing wall. In some embodiments, the coating does penetrate into the tubing wall to varying depths of penetration (Dₚ) into the wall, with non-limiting varying Dₚ examples shown in FIG. 2 b), c), and d). It is noted that these figures illustrate uniform penetration of the coating material into the wall; however, the disclosure is not limited thereto and in some embodiments, the tie layer coating is non-uniformly distributed through the wall thickness (where the values below may refer to maximum depth values or average depth values).

Again, in preferred embodiments, the thermoplastic coating penetrates into the wall to a depth of less than 100% the wall thickness, i.e., such that no thermoplastic material from the thermoplastic coating penetrates through to the ID of the tubing. For example, in various embodiments, the thermoplastic material coating penetrates into the wall to a depth of 90% or less, 80% or less, 70% or less, 60% or less, 50% or less, 40% or less, 30% or less, 20% or less, 10% or less, or 5% or less of the wall thickness (from the OD toward the ID of the tubing). In some embodiments, the depth to which the thermoplastic coating material permeates and/or the depth at which the coating material anchors into the wall of the ePTFE liner can be influenced by the size of the IND of the ePTFE, i.e., as IND increases, the Dₚ to which the coating material permeates into the tubing wall can increase (all other parameters being equal). In some embodiments, the penetration of thermoplastic coating material into the tubing wall is uniform; in some embodiments, the penetration of thermoplastic coating material into the tubing wall is non-uniform (and can vary, for example, along the length of the tubing and/or around the circumference of the tubing). Advantageously, in some embodiments, the inner surface (ID) of the tubing is completely free of thermoplastic coating material.

In some embodiments, the thermoplastic material coating shows good adhesion to the OD of the ePTFE tube, making it suitable to act as a tic layer to a jacket material that can be reflowed or otherwise applied over the outer diameter of the tube. Although not intending to be limited by theory, it is believed that the thermoplastic coating material permeates into open cells of ePTFE having a sufficiently large IND (as provided herein) to anchor the coating into the wall of the ePTFE tubing. An ePTFE tubing with a coating as described herein can, in some embodiments, be evaluated by taking a cross section of the tube and observing it using the SEM. SEM can be used to characterize the surface coating, e.g., by to determine its thickness as well as any defects present such as air pockets between the ePTFE liner tube and the surface coating. In some embodiments, minimal to no defects are present in an evaluated cross-section as observed by SEM.

In some embodiments, the thermoplastic material, its thickness and/or its depth of penetration into the ePTFE tubing walls impacts the physical properties of the resulting coated tubing (and those of any construction into which the coated tubing is incorporated). As described below, in some embodiments, the method of applying the thermoplastic coating can be modulated to achieve a desired thickness and/or depth of penetration, which can impact the physical properties (e.g., flexural stress and peel strength force values). Flexural stress of the coated ePTFE liners is directly affected by the durometer and coating thickness of the thermoplastic material coating. Since the durometer of the thermoplastic material relates to stiffness, as the durometer increases, the flexural stress of the coated ePTFE liner increases and therefore, the liner becomes stiffer. As coating thickness (which can be altered, e.g., via solution viscosity and residence time in solution as provided below) increases, the flexural stress of the coated ePTFE liner increases, thereby increasing stiffness.

Since ePTFE liners can be used to create more flexible catheters, it is advantageous in certain embodiments that the flexural stress of the coated ePTFE liner does not increase to a degree that using ePTFE as a catheter liner is no longer beneficial for flexibility. Additionally, peel strength of the coated ePTFE liner is affected by the ability of the thermoplastic material coating to anchor into the pores of the ePTFE liner tubing wall. The Dₚ of the thermoplastic material coating into the wall of the coated ePTFE liner can be impacted, e.g., by the durometer of the thermoplastic material and the temperature of the thermoplastic material solution during application to the ePTFE tubing (as well as by the IND of the ePTFE, as described above). Therefore, these properties are advantageously considered and selected to create an ePTFE liner with a thermoplastic material coating that penetrates deep enough into the pores of the ePTFE liner to anchor well and exhibit sufficiently high peel strength for catheter construction, but that does not totally permeate into the ID of the ePTFE liner and unnecessarily increase the coefficient of friction on the ID.

Methods of preparing the coated ePTFE tubings described herein can vary. In some embodiments, a thermoplastic material coating is applied to an ePTFE tubing by contacting (e.g., dip coating) an ePTFE tubing in a thermoplastic tie layer solution to apply the thermoplastic material to the outer surface of the tubing. For example, in some embodiments, an ePTFE tubing is placed on a mandrel or other support and dipped, e.g., at an angle, into a solution of the thermoplastic material. The solution of the thermoplastic material can comprise or can consist essentially of the thermoplastic polymer or polymers in any solvent in which the polymer or polymers are substantially soluble. In some embodiments, the solvent can comprise an alcohol, e.g., methanol, ethanol, n-butanol, isopropanol, and the like. In some embodiments, the solution can comprise tetrahydrofuran (THF). The concentration of the thermoplastic material in the solvent (and thus the viscosity of the solution) can vary and is not particularly limited; concentration and viscosity can impact the process and the physical properties of the resulting coated ePTFE tubing, as described herein below. In some embodiments, the thermoplastic material solution is at room temperature. In some embodiments, the solution is heated; the heating can provide the thermoplastic material solution at a temperature between room temperature and about 10° C. below the solvent boiling point.

The time for which the ePTFE tubing is in contact with the thermoplastic material solution can vary. In some embodiments, the contact time is about 5 seconds or longer, about 10 seconds or longer, about 20 seconds or longer, or about 30 seconds or longer. In some embodiments, the contact time is about 10 seconds to about 60 seconds, although the disclosure is not limited thereto and in some embodiments, the contact time can be considerably longer. In some embodiments, the ePTFE tubing can be contacted a single time with the solution and in some embodiments, the ePTFE tubing can be contacted two or more times with the thermoplastic material solution (e.g., with a drying step between each contact as described below).

Following contacting the ePTFE tubing with a solution of the thermoplastic material, the resulting tubing is dried. Drying can include, e.g., drying the tubing under ambient conditions (e.g., air temperature/pressure) and/or drying at elevated temperature, such as in a circulating air oven. Where the coated liner is dried at elevated temperature, the elevated temperature is generally a temperature at or above the boiling point of the solvent of the thermoplastic material solution. The drying is generally sufficient to remove at least a majority of the solvent from the coated ePTFE tubing such that the final coated ePTFE comprises little to no residual solvent (e.g., less than about 5% by weight of solvent; less than about 4% by weight of solvent, less than about 3% by weight of solvent, less than about 2% by weight of solvent, less than about 1% by weight of solvent, less than about 0.5% by weight of solvent, or less than about 0.1% by weight of solvent. In some embodiments, the coated ePTFE tubing consists essentially of ePTFE and the thermoplastic material.

Advantageously, once the ePTFE tubing is dried, the thermoplastic material advantageously remains as a coating on the outer surface of the ePTFE and, in some embodiments, within the walls of the ePTFE tubing (to a depth not to exceed the wall thickness, as described herein). The thermoplastic solution can permeate into the open cells of ePTFE with an IND as described herein to anchor the thermoplastic coating material into the walls of the ePTFE once the solution has dried. In some embodiments, the depth of penetration and/or the depth at which the tie layer material anchors into the wall of the ePTFE liner can be influenced by the size of the IND of the ePTFE, i.e., as IND increases, the depth at which the tie layer permeates into the tube surface increases.

In some embodiments, the tie layer coating can show sufficient adhesion to the OD of the ePTFE tubing so as to make it suitable to act as a tie layer, e.g., to a jacket material that can be reflowed or otherwise applied over the outer diameter of the tube. Suitable adhesion can be measured, e.g., as described in the Examples below, and in some embodiments, suitable adhesion can be demonstrated by average peel strength values of at least 0.1 N or at least 0.2 N (e.g., 0.1 to 0.5 N), e.g., measured by the peel test outlined below.

In some embodiments, the physical properties (e.g., the flexural stress and peel strength force values) of the coated ePTFE liner can be customized to some extent by modulating the viscosity of the solution and/or the time of contact between the ePTFE liner and the thermoplastic material solution. The viscosity and time relate to the thickness of the thermoplastic material coating on the ePTFE liner. Viscosity of the solution can be affected by the temperature of the thermoplastic material layer solution and the durometer of the thermoplastic material. In some specific embodiments, the thermoplastic material coating is applied in the form of a solution that is 5% PEBA 55D by weight in n-butanol. In some specific embodiments, the thermoplastic solution is 5% PEBA 25D by weight in n-butanol. In some specific embodiments, the thermoplastic solution is 5% TECOFLEX™ 93A polyurethane by weight in n-butanol. In some specific embodiments, the thermoplastic solution is 3% TECOFLEX™ 93A polyurethane in n-butanol. In some embodiments, the thermoplastic solution is 8% TECOTHANE™ polyurethane in tetrahydrofuran.

In some embodiments, the thermoplastic tie layer coating can be customized to create desired flexural stress and peel strength force values. Much of this customization can be provided by modulating the viscosity of the tic layer solution and/or the time the ePTFE tubing spends in the thermoplastic tie layer solution, all which directly relate back to the thickness of the tie layer coating on the ePTFE tubing. Viscosity of the tie layer solution can be affected by the temperature of the tie layer solution and durometer of the tie layer. As the temperature of the tie layer solution decreases, the solution viscosity increases. When dip coating an ePTFE liner in a more viscous tic layer solution, a thicker coating of the solution will be applied to the tube and the solution will not travel as deeply into the pores of the ePTFE tubing wall. When dip coating an ePTFE liner in a less viscous tic layer solution, a thinner coating of the solution will be applied to the tube and the solution will anchor deeper into the pores and wall of the ePTFE tubing. Similarly, the durometer of the tie layer thermoplastic material affects the viscosity of the tie layer solution, as higher durometer thermoplastics have a higher viscosity in solution at a given temperature. Therefore, at a set temperature, an ePTFE tubing dipped in a higher durometer tic layer thermoplastic material solution will have a thicker coating of tie layer compared to an ePTFE tubing dipped in a lower durometer tic layer thermoplastic material. Additionally, the length of time the ePTFE tubing stays in tic layer solution affects the thickness of the tic layer on the ePTFE tubing. This can either be demonstrated through having a longer residence time in the tic layer solution within a dip coating process or by multiple dip coating sessions between periods of drying. As the length of time spent in the tie layer solution increases, the thickness of the tic layer on the ePTFE tubing also increases. Thus, the tic layer coating and its properties can be controlled largely via coating thickness by setting specific parameters for tie layer durometer, tie layer solution temperature, and the residence time of the ePTFE tubing in the tie layer solution.

These parameters, especially as they relate to tic layer coating thickness, have a significant impact on the properties of the coated ePTFE liner. Flexural stress of the coated ePTFE liners is directly affected by the durometer and coating thickness of the tic layer. Since the durometer of the thermoplastic tic layer relates to stiffness, as the durometer increases, the flexural stress of the coated ePTFE liner increases and therefore becomes stiffer. As coating thickness (altered via solution viscosity and residence time in solution) increases, the flexural stress of the coated ePTFE liner increases, thereby increasing stiffness. Since ePTFE liners can be used to create more flexible catheters, it is imperative that the flexural stress of the coated ePTFE liner does not increase to a degree that using ePTFE as a catheter liner is no longer beneficial for flexibility. Additionally, peel strength of the coated ePTFE liner is affected by the ability of the tie layer to anchor into the pores of the ePTFE liner wall. The depth of which the tie layer travels into the wall of the ePTFE liner is impacted by the durometer of the tic layer material and the temperature of the tic layer solution during application to the ePTFE tubing. Therefore, these properties must be carefully considered to create an ePTFE liner with a tie layer that travels deep enough into the pores of the ePTFE liner to exhibit sufficiently high peel strength for catheter construction but that does not totally permeate into the ID of the ePTFE liner and unnecessarily increase the coefficient of friction on the ID. Therefore, the variables of the dip coating process to create coated ePTFE liners in some embodiments are balanced carefully so that a given ePTFE liner exhibits the properties desired to perform as a suitable ePTFE liner for catheters.

For building catheters using coated liners as described herein, instruments such as the Beahm 810A vertical laminator can be used. Coated ePTFE liners as described herein can be bonded with catheter jackets, e.g., using conventional means. For example, suitable catheter jackets include, but are not limited to, catheter jackets comprising PEBA, nylon, polyurethane, etc., and suitable heat shrink tubings include, but are not limited to, heat shrink tubings comprising PEBA, Nylon, FEP, PET, PTFE, etc.

Coated ePTFE liners as provided herein can, in some embodiments, be stretched with or without heat to reduce the wall thickness and then used to build catheters. The degree of stretching of the liners can affect some physical, mechanical, or thermal properties of the built catheters, such as lubricity, tenacity, modulus, etc. Typically, increasing the degree of stretching increases the axial orientation of the polymer, thereby increasing the modulus and tensile strength. As such, in some embodiments, coated ePTFE liners with at least some degree of axial orientation are provided.

In some embodiments, the coated ePTFE liners can be reinforced, e.g., braided with metallic or non-metallic wires/filaments/yarns before lamination with catheter jackets using braiding machines such as the Steeger Medical Braider. These types of braiding machines can be used to braid with different patterns, pick count, length, etc. using wires/filaments/yarns of various profiles and sizes. The change in braid pattern and pick count can affect the overall properties of the catheter, such as hoop strength, flexibility, torque response, etc. Further reinforcements, including polymeric and/or metal coiled, tubular, and/or fiber structures can be employed in certain catheter constructions comprising the coated ePTFE liners described herein.

### EXAMPLES

Aspects of the present disclosure more fully illustrated by the following examples, which are set forth to illustrate certain aspects of the present invention and are not to be construed as limiting thereof.

A Hitachi Model TM3030 Scanning Electron Microscope (SEM) was used to characterize the suitability of ePTFE liner tubes for use with a surface coating as well as to characterize the quality of the surface coating.

### Adhesion Testing

Adhesion between the base tube (here, ePTFE tubing) and the tie layer material may be tested by reflowing a jacket material over the coated tube and subjecting the final structure to a ‘bond test’. A method relying on the physical examination of a test specimen after conducting a ‘bond test’ was used to determine if an adequate bond was created between the outer surface of coated ePTFE liner tube and the inner surface of an overlying PEBA outer jacket tube or a PEBA tube acting as a combined outer jacket and heat shrink tube. This method involves fabricating a short section of an unreinforced catheter shaft, e.g., approximately 10 to 15 cm in length. This allows for a comparison of the bond that is formed between the outer surface of the coated ePTFE liner tube and the inner surface of the outer jacket tube or combined outer jacket and heat shrink tube.

Preparation of the bond test specimens began by fabricating an unreinforced catheter shaft subassembly for each Example that was tested.

Fabrication began by hand stretching a tie layer-coated ePTFE liner tube as described herein. A tie layer-coated ePTFE liner tube with an inner diameter of about 1.78 mm and an average wall thickness of about 0.0911 mm was hand stretched over a glass-filled mandrel having an outer diameter of about 1.75 mm. This stretching step forces the coated ePTFE liner tube to “draw down” and fit snugly over the mandrel, resulting in an unreinforced catheter shaft subassembly. A 2.5 cm-long thin strip of polyimide tape was wrapped around one end of the subassembly (i.e., on the outside surface of the coated ePTFE liner to prevent bonding between the outer surface of the coated ePTFE liner and the inner surface of the combined outer jacket and heat shrink tube where the foil or tape was applied. The inclusion of the polyimide tape strip serves to provide a “tab” from which to peel apart the inner coated ePTFE liner and the outer jacket/heat shrink tube after the build was complete.

A PEBA tube having an expanded inner diameter of about 2 mm, a wall thickness of about 0.076 mm, and a recovered inner diameter of 1.9 mm was utilized for a combined outer jacket and heat shrink tube. The glass-filled PTFE mandrel, coated ePTFE liner, and PEBA outer jacket/heat shrink tube assembly was suspended vertically from an oven rack using clips and heated in a standard laboratory gravity convection oven to finalize the build. After heating the specimens for the time and temperature required, they were allowed to cool to room temperature and subsequently removed from the glass-filled PTFE mandrel. The unreinforced catheter shaft specimen was slit longitudinally on one side using a razor blade and flattened to give a ‘rectangular’ shape. The bond test was performed by grasping in one hand the tab end of the coated ePTFE liner (i.e., the portion that was underneath the polyimide film) and the polyimide film tab attached to the PEBA tube in the other hand and slowly pulled in opposite directions for approximately 5 cm.

After peeling, careful examination of the section of coated ePTFE liner that was peeled away from the PEBA outer jacket tube can aid in determining if an adequate bond was created between the outer surface of the coated ePTFE liner and the inner surface of the PEBA outer jacket tube. For coated ePTFE liner tubes with a thermoplastic tie layer adhered to the outer surface of the ePTFE liner, it is understood that combining such technology will substantially increase the bond strength between the liner and outer jacket. It can be inferred through this test that an inadequate bond was formed during the heating step if the coated ePTFE liner is easily peeled away from the PEBA outer jacket tube (i.e., there is little or no resistance to peeling the layers apart). However, if the PEBA outer jacket tube did adequately bond to the outer surface of the coated ePTFE liner during the heating step, the outer surface of the coated ePTFE liner may pucker or rip away from the PEBA outer jacket tube. It can be inferred through this test that an adequate bond was formed during the heating step if it is difficult to peel the coated ePTFE liner away from the PEBA outer jacket tube (i.e., there is some resistance or substantial resistance to peeling the layers apart). The peel strength force values were averaged between replicates and are displayed in Table 1.

### Coefficient of Friction Testing

The Coefficient of Friction (COF) was obtained using a TA instruments Discovery Hybrid Rheometer (DHR-3) with the tribo-rheometer accessory to determine the tribological properties of the ID of both coated and uncoated ePTFE liners. The samples were prepared by attaching three tubing sections of 5 mm×16.5 mm, each, to the three teeth of the half-ring for use with a Ring-On-Plate tribo-rheometry fixture. The ring with mounted samples was then attached to the ring-on-plate upper-geometry holder and lowered to have the samples contact a mirror-finish stainless steel plate at the specified axial force. A tribological test was performed at room temperature (23° C.) from sliding speeds of 750 μm/s to 7650 μm/s under an axial load of IN. An additional tribological test was performed at 40° C. with a 5-minute dwell time at 40° C. from sliding speeds of 750 μm/s to 7650 μm/s under an axial load of IN. Minimum COF over the stated range in sliding speed was calculated by the TA instruments TRIOS software v4.3. At least 3 samples were tested for each loading and temperature; the average value at room temperature is listed in Table 1.

### Tensile Property Testing

An Instron 5965 dual column mechanical tester running Bluehill 3 v3.73.4823 operating software was used to determine the tensile properties of the ePTFE tubes. The test was performed at a rate of 5.08 cm/min using a 20 lbf load cell attached to pneumatic grips with smooth face inserts set to a 5.08 cm gage length. During analysis, for tensile modulus determination of the first segment, a straight line was fitted between 0 to 5% elongation/tensile strain. For tensile modulus determination of the second segment, a straight line was fitted between 10 to 50% elongation/tensile strain.

Three-point bend data was obtained using a TA instruments Q800 Dynamic Mechanical Analysis (DMA) using tubular geometry to determine the flexural properties of uncoated and coated ePTFE liners. The ePTFE tubes were cut to 10 mm lengths and testing was performed with a span length of 5 mm and preload of 0 N. At least 3 samples were tested using this process. DMA data was imported into TA instruments TRIOS software v4.3, and the averages of flexural stress at a given strain percentage are listed in Table 1.

### Imaging

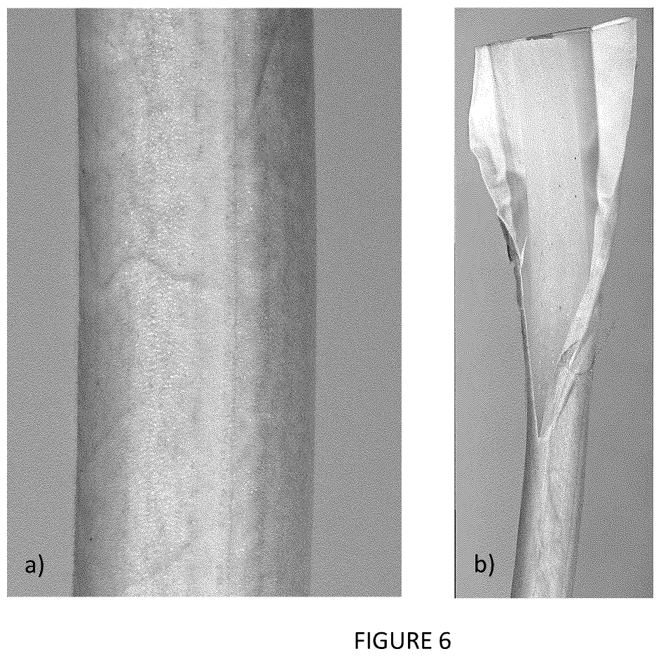

A Keyence VHX-5000 digital microscope was utilized for further imaging, e.g., to determine if the tie layer coating permeated through the inner diameter of the ePTFE liner. By using a green dyed PEBA solution to coat the ePTFE liner, images could be obtained comparing the inner surface of the ePTFE liner versus the outer surface of the coated ePTFE liner. If there is little to no green showing on the ID of the ePTFE liner, it can be determined that the tie layer did not significantly flow through to the inner surface of the tube. See FIG. 6. The Keyence microscopy images provided in FIG. 6 display the apparent absence of PEBA tie layer leaching/permeating through to the inner diameter of the ePTFE liner produced with the same process as Example 3; the only difference from Example 3 is that Example 3 used unpigmented PEBA.

### Catheter Testing

Built catheters and catheter components such as liners can be tested using an interventional device testing equipment such as the IDTE3000 from MSI which can measure and record device performance features such as push force, flexibility, torqueability, etc. Multiple test cycles can be performed on the same sample to measure change in torque/push force due to potential abrasions on the inside surface from repeated testing.

### Comparative Example 1

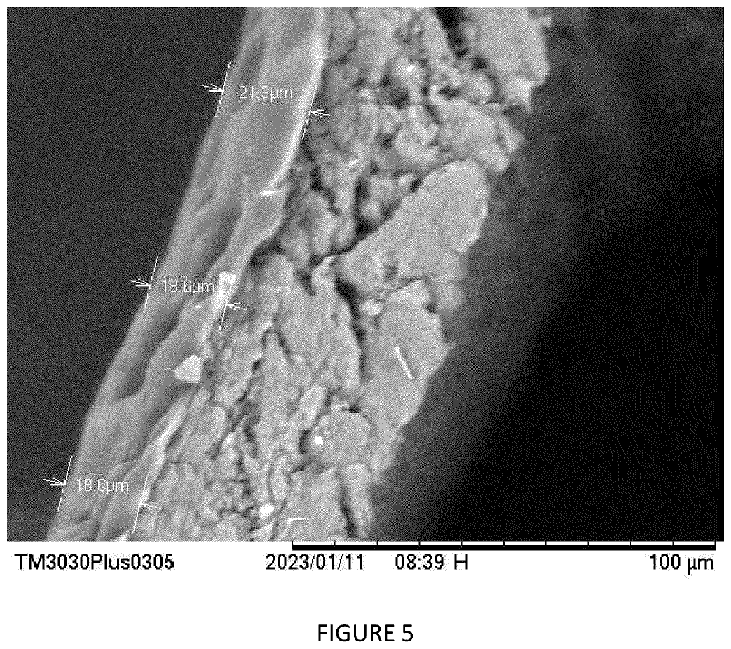

An ePTFE tube with a 2.10 mm ID, 0.44 mm wall thickness and approximately 3 micron IND was coated with 5% Tecoflex™ 93A dissolved into n-butanol. A 13 cm long ePTFE tube was slid over a mandrel and dipped into the solution at 25° C. so that the outer surface was completely coated after 10 seconds. The coated tube was hung vertically to dry in air for 60 minutes. After drying, the coated tube was cut into cross sections and subjected to SEM analysis. See FIG. 5. It was noted that the Tecoflex™ solution lay on top of the outer surface with a 10-20 μm thickness without having penetrated through the wall into the ID.

### Comparative Example 2

The ePTFE tube of Comparative Example 1 was dipped into the Tecoflex™ Solution of Comparative Example 1 at 80° C. Upon SEM analysis, it was noted that the coating had a thickness of about 2-10 μm (i.e., thinner than the coating of Comparative Example 1), but that it had also not penetrated into the wall into the ID.

### Example 1

A 13 cm ePTFE tube with a 0.041 mm wall thickness and 40-60 micron IND was dipped into a 5% Tecoflex™ 93A solution for 10 seconds in the manner of Comparative Example 1 at a temperature of 80° C. The tube was then hung vertically to dry at room temperature for 60 minutes and cut into cross sections and photographed under SEM. The tube had a 5-20 micron layer of Tecoflex™ on the surface of the ePTFE and had penetrated into the wall without penetrating through the wall into the ID.

### Example 2

A 13 cm long ePTFE tube with 0.60 mm ID, 0.041 mm wall thickness and 40-60 micron IND was dipped into a 5% PEBA 55D solution at a temperature of 80° C. in the manner of Example 1. The tube was then hung vertically to dry at room temperature for 60 minutes and cut into cross sections and photographed under SEM. The tube had a 5-20 micron layer of PEBA on the surface of the ePTFE and had penetrated into the wall without penetrating through the wall into the ID (e.g., adsorbed to the wall surface without having adsorbed/diffused completely through the wall).

### Example 3

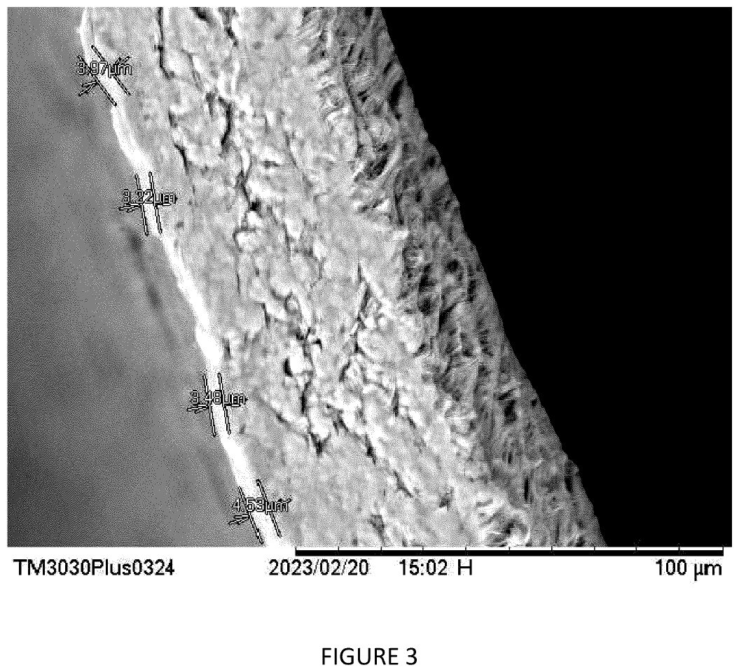

A 1 meter long ePTFE tube with a 1.78 mm ID, 0.051 mm wall thickness and 20-30 micron IND was dipped into a 5% PEBA 55D solution at a temperature of 80° C. in the manner of Comparative Example 1. The tube was then hung vertically to dry at room temperature for 60 minutes and placed into standard laboratory gravity convection oven at 177° C. to allow the PEBA 55D coating to settle into the pores. The coated ePTFE tube was cut into cross sections and photographed under SEM (see FIG. 3). The tube had a 3-10 micron layer of PEBA on the surface of the ePTFE and the PEBA layer had penetrated into the wall without penetrating through the wall into the ID (e.g., adsorbed to the wall surface without having adsorbed/diffused completely through the wall).

Data from testing on tubing from Example 3 is displayed below.

TABLE 1
Properties of ePTFE liners.

| | ePTFE liner, | ePTFE liner with |
|---|---|---|
| Property | uncoated | PEBA 55D Tie Layer |
| Average peel strength force (N) | — | 0.25 |
| Coefficient of friction of inner | 0.090 | 0.086 |
| diameter at 23° C. | | |
| Flexural stress @ 0.5% strain | N/A* | 0.004 |
| (MPa) | | |

*During testing, the sample did not meet the minimum required force, which is 0.001N. Therefore, there was not enough signal to measure and get a true flex modulus on the sample.

### Example 4

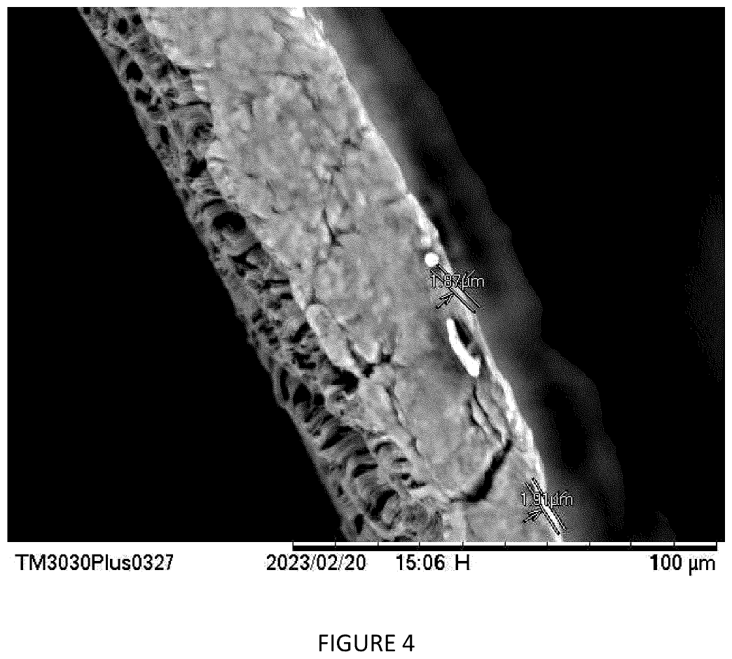

A 100 mm long ePTFE tube with a 1.78 mm ID, a 0.051 mm wall thickness and 20-30 micron IND was dipped into a 5% PEBA 25D solution at a temperature of 80° C. for 20 s in the manner of Example 1. The tube was then hung vertically to dry at room temperature for 60 minutes and cut into cross sections and photographed under SEM (see FIG. 4). The tube had a 1-5 micron layer of PEBA on the surface of the ePTFE and had penetrated somewhat into the wall of the tube without penetrating through the wall into the ID (e.g., adsorbed to the wall surface without having adsorbed/diffused completely through the wall).

What is claimed is:

1. A coated tubing, comprising:
an expanded polytetrafluoroethylene (ePTFE) tubing with an inner surface and an outer surface and a wall with a thickness; and
a thermoplastic tie layer coating on at least a portion of the outer surface.

2. The coated tubing of claim 1, wherein the thermoplastic tie layer coating is on substantially all of the outer surface.

3. The coated tubing of claim 1, wherein the thermoplastic tie layer coating permeates into the wall from the outer surface through the wall of the ePTFE tubing to a depth of 80% or less of the wall thickness.

4. The coated tubing of claim 3, wherein the thermoplastic tie layer coating permeates into the wall from the outer surface through the wall of the ePTFE tubing to a depth of 60% or less of the wall thickness.

5. The coated tubing of claim 4, wherein the thermoplastic tie layer coating permeates into the wall from the outer surface through the wall of the ePTFE tubing to a depth of 50% or less of the wall thickness.

6. The coated tubing of claim 1, wherein the inner surface comprises no thermoplastic tie layer coating.

7. The coated tubing of claim 1, wherein the thermoplastic tie layer coating has an average coating thickness from the outer surface outwards of 1 μm or greater.

8. The coated tubing of claim 1, wherein the thermoplastic tie layer coating has a durometer hardness between 93A and 55D.

9. The coated tubing of claim 1, wherein the thermoplastic tie layer coating comprises or consists essentially of one or more of a polyether block amide, a polyurethane, a nylon, or a derivative or copolymer thereof.

10. The coated tubing of claim 3, wherein the thermoplastic tie layer coating is non-uniformly distributed throughout the wall thickness.

11. The coated tubing of claim 1, wherein the ePTFE tubing has an inner diameter of 0.6 mm or greater.

12. The coated tubing of claim 1, wherein the ePTFE tubing has an average wall thickness of 0.03 mm or greater.

13. The coated tubing of claim 1, wherein the ePTFE tubing has an internodal distance (IND) of greater than 5 μm.

14. A catheter comprising the coated tubing of claim 1, configured such that the catheter has a catheter inner surface corresponding to the inner surface of the ePTFE tubing.

15. The catheter of claim 14, further comprising a reinforcing layer on an outer surface of the coated tubing (e.g., a braided or coiled wire).

16. The catheter of claim 14, further comprising a catheter jacket.

17. A catheter assembly comprising:
an expanded polytetrafluoroethylene (ePTFE) liner with an inner surface and an outer surface and a wall thickness; and
a thermoplastic tie layer coating with an average coating thickness of 1 μm or greater and a durometer hardness between 93A and 55D disposed on the outer surface of the ePTFE liner to give a coated liner,
wherein the ePTFE liner has a bonding affinity to the thermoplastic tie layer coating.

18. The catheter assembly of claim 17, wherein the thermoplastic tie layer coating is on substantially all of the outer surface.

19. The coated tubing of claim 1, wherein the thermoplastic tie layer coating permeates into the wall from the outer surface through the wall of the ePTFE tubing to a depth of 80% or less of the wall thickness.

20. The catheter assembly of claim 17, wherein the inner surface comprises no thermoplastic tie layer coating.
